(19) United States
(12) Patent Application Publication
(10) Pub. No.: US 2009/0271904 A1
(43) Pub. Date: Nov. 5, 2009

(54) Headband device and method of using the same

(76) Inventor: Richard Joseph Bentley, Westport, CT (US)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/150,764

(22) Filed: May 1, 2008

(51) Int. Cl. A61F 9/04

(52) U.S. Cl. 2/15; 2/173; 2/206

(57) ABSTRACT

A headband device permits a user to rest, even sleep, snugly fastened to the headrest of the seat in which he or she is seated. A method of using the headband device is also disclosed.

### BACKGROUND OF THE INVENTION

1. Technical Field

The present invention relates generally to a headband worn around the head and, more particularly, to a headband which allows a wearer to rest, even sleep, snugly fastened to the headrest of the chair or seat in which he or she is seated.

2. Description of the Background Art

Persons attempting to sleep sitting up risk straining their necks or being unable to sleep because their head keeps falling or jerking down. Passengers of commercial aircraft, in particular, those seated in Economy Class where space is limited and seats recline only slightly are particularly vulnerable to these risks. It would be useful to stabilize the head of someone attempting to sleep while seated upright.

### SUMMARY OF THE INVENTION

It is an object of the present invention to provide a novel method and headband for the purpose of stabilizing the head of someone attempting to sleep while seated upright.

It is also an object to provide such a method and headband which may be used with any seat the back of which is tall enough to reach head level.

Still another object is to provide such a method and headband which, while the user's head is stabilized, there is no danger of harm resulting from the headband if there is any sudden movement of the chair or even the plane in which user may be traveling.

A further object is to provide such a headband which will be comfortable to wear because of the use of soft elasticized materials from which it will be made.

It has now been found that the foregoing and related objects can be readily attained in a device for use in temporarily securing the head of a user to a seat having a headrest thereon, comprising (a) a headband incorporating an elasticized material and having a first portion of a mating hook and clinging pile material attached to at least a rear portion of the headband, the headband with the first portion thereon being dimensionally sized to be received on and extend entirely around the head of the user and cover the eyes thereof; and (b) a second portion of the mating hook and clinging pile material having means thereon for temporarily securing the second portion of the mating hook and clinging pile material to the headrest of the seat, wherein, once the second portion of the mating hook and clinging pile material is temporarily secured to the headrest of the seat utilizing the securing means, the first portion of the mating hook and clinging pile material attached to the headband is adapted to be temporarily secured to the second portion thereby temporarily securing the head of the user of the headband to the headrest of the seat.

Desirably, the first portion of the mating hook and clinging pile material is a strip of hook material attached to the rear portion of the headband.

According to the invention, the securing means for temporarily securing the second portion of the mating hook and clinging pile material to the headrest of the seat is provided by a sticky back on the second portion of the mating hook and clinging pile material. The second portion of the mating hook and clinging pile material is a strip of clinging pile material.

Conveniently, the elasticized material is an elasticized cotton material or other soft absorbent material.

In practicing the method of the invention, the second portion of the mating hook and clinging pile material is temporarily secured to the headrest of the seat utilizing the securing means. Once the headband is extended entirely around the head of the user and covering the eyes thereof, the first portion of the mating hook and clinging pile material attached to at least the rear portion of the headband can be temporarily secured to the second portion thereby temporarily securing the head of the user of the headband to the headrest of the seat.

The invention will be fully understood when reference is made to the following detailed description taken in conjunction with the accompanying drawings.

### BRIEF DESCRIPTION OF THE DRAWINGS

FIG. 1 is a perspective view of the headband of the present invention;

FIG. 2 is a perspective view of the pile attachment portion of the headband of this invention;

FIG. 3 is a front view of the headband of this invention on the head of a user;

FIG. 4 is side view of the headband of this invention on the head of a user;

FIG. 5 is a rear view of the headband of this invention on the head of a user; and

FIG. 6 is a perspective view of a user seated in a commercial aircraft seat and utilizing the present invention.

### DETAILED DESCRIPTION OF THE INVENTION

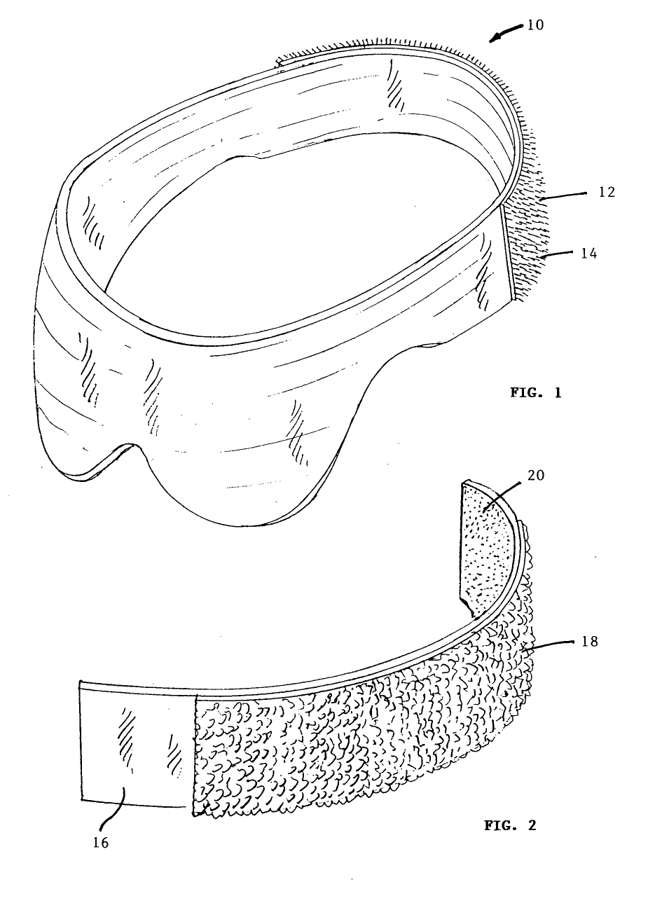

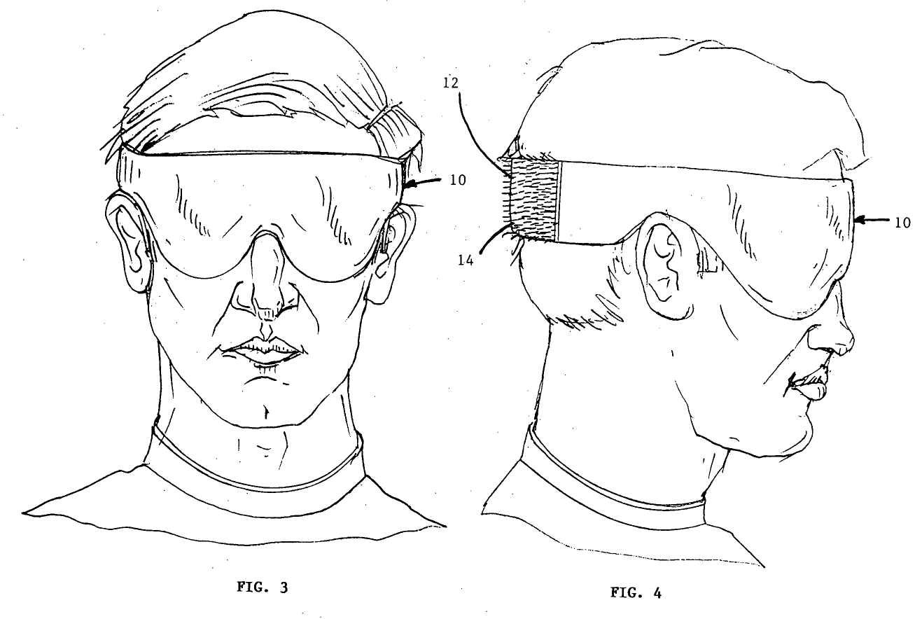

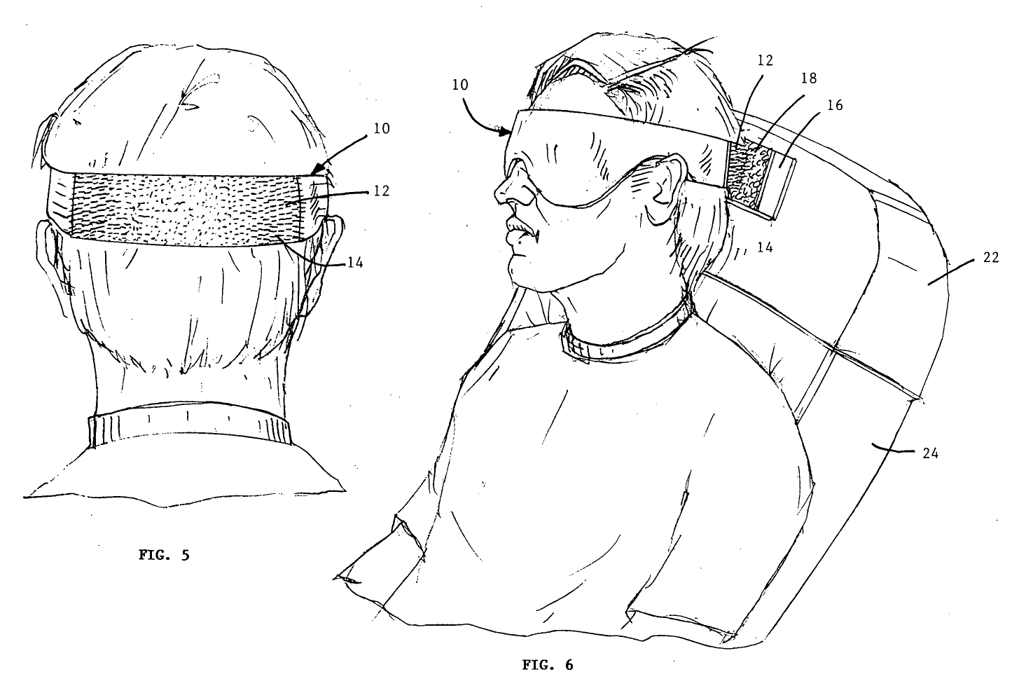

Turning to FIG. 1 of the drawings, therein illustrated is a headband which is generally designated by the numeral 10 and made in accordance with the present invention. The headband is made of elasticized cotton or other soft absorbent material and shaped to go over the eyes and around the nose of the user. The shape and manner of wearing the headband 10 may be seen in FIGS. 3 through 5. The back of the headband 10, i.e. that part which will touch the headrest of the seat in which the user is sitting, consists of or is fitted with the “hook” side 12 of a Velcro strip 14.

As shown in FIG. 2, the headband is accompanied by a longer strip 16 of the “clinging pile” side 18 of Velcro with a sticky back 20. As shown in FIG. 6, the strip 16 can be easily stuck temporarily to a headrest 22 of a seat 24 and then can be used to form a bond with the headband 10 itself.

Once the clinging pile 18 of Velcro is stuck to the headrest 22 at the appropriate level and location, the user will slip the headband over his or her head and simply lean back to make the connection between the two sides 12, 18 of Velcro. This will have the effect of stabilizing the head in a comfortable position, making it possible for the user to doze or sleep without having their head falling forward or leaning to the side. Without the device of the present invention, a person attempting to sleep sitting up risks straining the neck or being unable to sleep because the head keeps falling or jerking down. In the case of airplane passengers, should the seat recline in which the headband wearer is sitting, the user will be more comfortable still.

While the user's head is stabilized, there is no danger of harm resulting from any sudden movement of the seat or even the plane in which he or she may be traveling because the sides 12,18 will separate if modest force is applied. The user may also simply lift the headband 10 up to remove. The headband 10 and related Velcro strip 16 may be removed after use and retained for use in the future.

Thus, it can be seen from the foregoing specification and attached drawings that the device and method of the present invention facilitates the rest, even sleep of travelers or other people seated upright in chairs that do not recline or recline only slightly by stabilizing their heads. The device provides stability to the head of the user who is forced or attempting to rest or sleep in an upright position.

1-6. (canceled)

7. A device for use in temporarily securing the head of a user to a seat having a headrest thereon, comprising:
(a) a headband incorporating an elasticized material and having a first portion of a mating hook and clinging pile material attached to at least a rear portion of the headband, the headband with the first portion thereon being dimensionally sized to be received on and extend entirely around the head of the user and cover the eyes thereof; and
(b) a second portion of the mating hook and clinging pile material having means thereon for temporarily securing the second portion of the mating hook and clinging pile material to the headrest of the seat, wherein, once the second portion of the mating hook and clinging pile material is temporarily secured to the headrest of the seat utilizing the securing means, the first portion of the mating hook and clinging pile material attached to the headband is adapted to be temporarily secured to the second portion thereby temporarily securing the head of the user of the headband to the headrest of the seat.

8. The device according to claim 7, wherein the first portion of the mating hook and clinging pile material is a strip of hook material attached to the rear portion of the headband.

9. The device according to claim 7, wherein the securing means for temporarily securing the second portion of the mating hook and clinging pile material to the headrest of the seat is provided by a sticky back on the second portion of the mating hook and clinging pile material.

10. The device according to claim 9, wherein the second portion of the mating hook and clinging pile material is a strip.

11. The device according to claim 10, wherein the strip is a strip of clinging pile material.

12. The device according to claim 7, wherein the elasticized material is an elasticized cotton material or other soft absorbent material.

13. A method for temporarily securing the head of a user to a seat having a headrest thereon, comprising the steps of:
(a) providing a headband incorporating an elasticized material and having a first portion of a mating hook and clinging pile material attached to at least a rear portion of the headband, the headband with the first portion thereon being dimensionally sized to be received on and extend entirely around the head of the user and cover the eyes thereof;
(b) providing a second portion of the mating hook and clinging pile material having means thereon for temporarily securing the second portion of the mating hook and clinging pile material to the headrest of the seat;
(c) temporarily securing the second portion of the mating hook and clinging pile material to the headrest of the seat utilizing the securing means;
(d) extending the headband entirely around the head of the user and covering the eyes thereof; and
(e) temporarily securing the first portion of the mating hook and clinging pile material attached to at least the rear portion of the headband to the second portion thereby temporarily securing the head of the user of the headband to the headrest of the seat.

14. The method according to claim 13, wherein the first portion of the mating hook and clinging pile material is a strip of hook material attached to the rear portion of the headband.

15. The method according to claim 13, wherein the securing means for temporarily securing the second portion of the mating hook and clinging pile material to the headrest of the seat is provided by a sticky back on the second portion of the mating hook and clinging pile material.

16. The method according to claim 15, wherein the second portion of the mating hook and clinging pile material is a strip.

17. The method according to claim 16, wherein the strip is a strip of clinging pile material.

18. The method according to claim 13, wherein the elasticized material is an elasticized cotton material or other soft absorbent material.
